(19) United States
(12) Patent Application Publication
(10) Pub. No.: US 2022/0218907 A1
(43) Pub. Date: Jul. 14, 2022

(54) Medical Delivery Device

(71) Applicants: Deborah Lynn Fuller, Seattle, WA (US); James Thomas Fuller, Seattle, WA (US); Orlance, Inc., Seattle, WA (US)

(72) Inventors: Hannah Elise Frizzell, Seattle, WA (US); Kristyn Rene Aalto, Kingston, WA (US); Amanda Kay Woodcock, Seattle, WA (US); Dennis Earl McCabe, Seattle, WA (US); Deborah Lynn Fuller, Bainbridge Island, WA (US); James Thomas Fuller, Bainbridge Island, WA (US)

(21) Appl. No.: 17/571,047

(22) Filed: Jan. 7, 2022

Related U.S. Application Data
(60) Provisional application No. 63/135,616, filed on Jan. 10, 2021.

(51) Int. Cl. A61M 5/30; A61M 5/20

(52) U.S. Cl. CPC A61M 5/30; A61M 5/2053

(57) ABSTRACT

Disclosed and claimed is a medical delivery device with a separable housing comprising a durable component and a disposable component. The durable component comprises a handle portion of the device that may be connected to a compressed gas source to allow for the input of pressurized gas into the device. The disposable component comprises a cartridge containing a plurality of doses of biological or non-biological material and a supersonic barrel through which the dose is propelled out of the device and into the subject. When all of the doses in the cartridge have been administered, or when the device is to be used with a different subject, the cartridge is removed, and a new cartridge attached to the durable component.

### CROSS REFERENCE TO RELATED APPLICATIONS

This application claims the benefit of U.S. Provisional Application No. 63/135,616, filed Jan. 10, 2021, which is incorporated by reference.

### GOVERNMENT SUPPORT STATEMENT

This invention was made with government support under Grant Number: R43AI140784 awarded by the National Institutes of Health. The government has certain rights in the invention.

### TECHNICAL FIELD

The subject matter described generally relates to the field of medical devices, and in particular, to a medical delivery device for delivering a vaccine, medication, treatment, or other biological or non-biological material into the epidermis or mucosal tissue of a subject.

### BACKGROUND

Medical delivery devices, such as gene guns, may be used to deliver biological or non-biological material into a subject by accelerating high-density particles to high speeds that allow for epidermal penetration of the material. The use of these devices enables effective delivery of the material while avoiding shortcomings associated with delivery via needle and syringe or jet injectors, including the risk of cross-contamination, accidental needle stick, bruising, or infection. However, conventional gene guns are limited in the maximum dose of gold particles that can be delivered into a single shot to achieve optimum cell viability and in vivo transfection efficiency. Moreover, these devices typically use an internally vented solenoid valve to control the flow of pressurized gas from the gas source to the barrel, resulting in an increased time from solenoid opening to achieve maximum device pressure than if such valve were externally vented. This increased time for solenoid opening on conventional devices reduces the consistency of delivery and penetration of the material between shots.

### BRIEF DESCRIPTION OF THE DRAWINGS

FIG. 1 illustrates a side view of a medical delivery device, according to one embodiment.

FIG. 2 illustrates external components of a housing of the medical delivery device of FIG. 1, according to one embodiment.

FIG. 3 illustrates an internal view of the medical delivery device of FIG. 1, according to one embodiment.

FIG. 4 illustrates a dose counter window of the medical delivery device of FIG. 1, according to one embodiment.

FIG. 5 illustrates a first exploded view of the separable housing of the medical delivery device of FIG. 1, according to one embodiment.

FIG. 6 illustrates a second exploded view of the separable housing of the medical delivery device of FIG. 1, according to one embodiment.

FIG. 7 illustrates the medical delivery device of FIG. 1 connected to a compressed gas source, according to one embodiment.

FIG. 8 illustrates example specifications of the supersonic barrel of the medical delivery device of FIG. 1, according to one embodiment.

FIG. 9 is a flow chart illustrating a method for operating the medical delivery device of FIG. 1, according to one embodiment.

### DETAILED DESCRIPTION

The Figures (FIGS.) and the following description describe certain embodiments by way of illustration only. One skilled in the art will readily recognize from the following description that alternative embodiments of the structures and methods may be employed without departing from the principles described. Reference will now be made to several embodiments, examples of which are illustrated in the accompanying figures. It is noted that wherever practicable similar or like reference numbers are used in the figures to indicate similar or like functionality.

### Overview and Benefits

Disclosed by way of embodiment is a medical delivery device with a separable housing comprising a durable component and a disposable component. The durable component (referred to throughout as a “reusable body”) comprises a handle portion of the device that may be connected to a compressed gas source to allow for the input of pressurized gas into the device. The disposable component comprises a cartridge containing a plurality of doses of biological or non-biological material and a supersonic barrel through which the dose is propelled out of the device and into the subject. When all of the doses in the cartridge have been administered, or when material is to be delivered to a different subject, the cartridge is removed, and a new cartridge attached to the reusable body. While the primary embodiment discussed herein contemplates a disposable cartridge, in another embodiment, a cylinder containing the doses of material is inserted into a durable cartridge such that the cartridge, in addition to the body, may be used for material delivery to multiple subjects.

The medical delivery device uses a high-velocity stream of gas to accelerate gold particles containing the material from the dose chamber through the supersonic barrel at speeds sufficient to penetrate cells. In one embodiment, the barrel comprises a primary expansion zone beginning at a first distal end of the barrel and an overexpansion zone beginning at an endpoint of a step separating the zones and ending at the second distal end of the barrel in an outlet nozzle that may be placed against the epidermis of a subject (e.g., against the subject's arm or other skin sites) for delivery of the material. Alternatively, the device may be used for mucosal tissue delivery of material into the subject (e.g., into the subject's eyelid, inner cheek, or tongue).

### Example Device

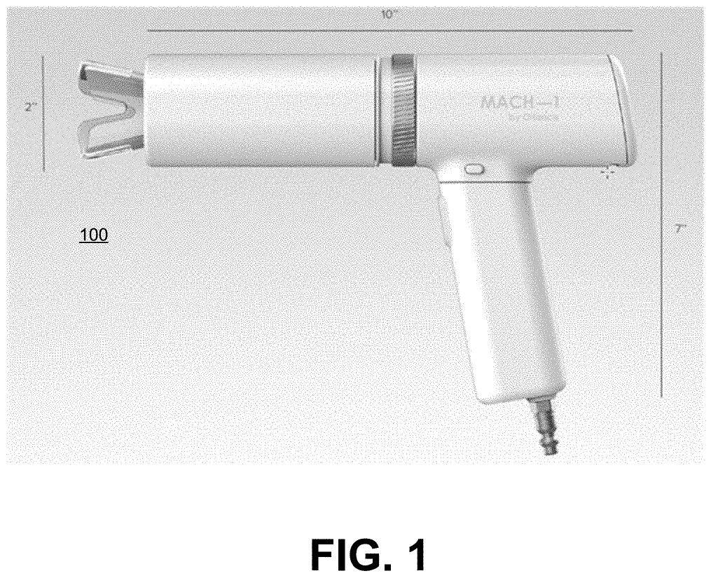

Figure (“FIG.”) 1 illustrates a side view of a medical delivery device 100, according to one embodiment. The medical delivery device 100 of FIG. 1 may be used to deliver vaccines, such as the COVID-19 vaccine or any other vaccine, medication, treatment, or biological or non-biological therapeutic payload (referred to throughout as “material”) loaded onto gold microparticles into the epidermis or mucosal tissue of a subject.

As discussed in more detail below, the device 100 has a separable housing comprising a reusable body on a handle side that is connected to an external compressed gas source and a disposable cartridge side that contains one or more doses of the material. In one embodiment, each cartridge may be used with a single subject (e.g., patient) and contain up to four doses of the material. Accordingly, the cartridge side may be removed (e.g., when the doses have all been administered or for a different subject), and the handle side may be re-used with a different cartridge containing the same or a different type of material for the same or a different subject.

In one embodiment, the medical delivery device 100 has a height of approximately seven inches from the top of the reusable body to the base of the handle and a width of approximately ten inches from a battery at the base of the reusable body to the end of the exterior of the device housing. The device 100 additionally includes a supersonic barrel for delivering the material into the epidermis or mucosal tissue of the subject. As shown in FIG. 1, the terminal end of the barrel extends outwardly from the device housing in a nozzle that may be placed against the subject when the device 100 is to be discharged. However, one of skill in the art will recognize that the device 100 may have a different form factor than in the embodiment described above. For example, in an alternate configuration, the reusable body of the device 100 may be wider and/or have a different shape to accommodate a larger solenoid valve inside the body.

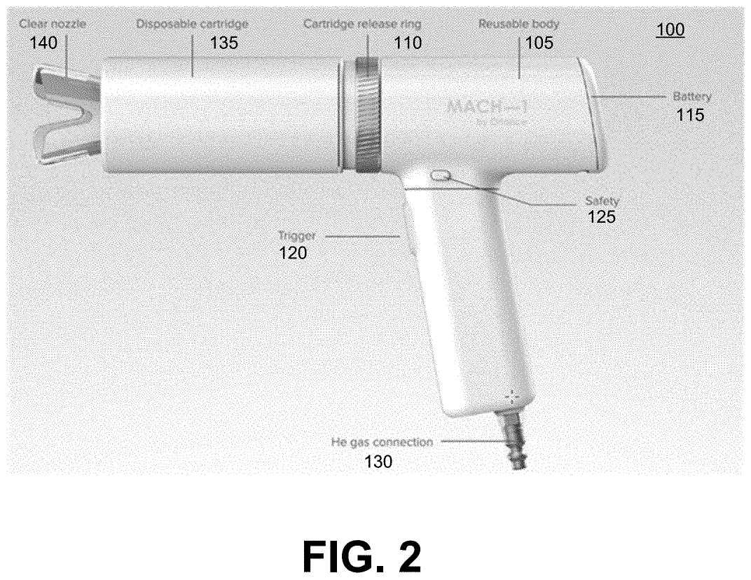

Turning now to FIG. 2, it illustrates external components of the housing of the medical delivery device 100 of FIG. 1, according to one embodiment. In the embodiment illustrated in FIG. 2, the medical delivery device 100 includes a reusable body 105 on a handle side of the device 100 and a disposable cartridge 135 on a barrel side of the device 100. The reusable body 105 includes a cartridge release ring 110, a battery 115, a trigger 120, a safety 125, and a gas connection 130. The disposable cartridge 135 includes a supersonic barrel 230 of which a nozzle portion 140 at the terminal end of the barrel 230 extends outwardly from an interior of the disposable cartridge housing. In other embodiments, the medical delivery device 100 contains different and/or additional elements. In addition, the functions may be distributed among the elements in a different manner than described.

The reusable body 105 may be coupled to the disposable cartridge 135 via the cartridge release ring 110. When the cartridge release ring 110 is engaged, it secures the handle side of the device 100 to the barrel side such that the internal components of the device 100 are operably coupled to allow for operation of the device 100 and the delivery of the material. In one embodiment, the cartridge release ring 110 may be turned, e.g., in a clockwise direction, to secure the cartridge 135 to the body 105 and turned in an opposite, e.g., counterclockwise, direction to decouple the cartridge 135 from the body 105, for example when replacing the disposable cartridge 135. One of skill in the art will recognize that other coupling means for securing the cartridge 135 to the body 105 may be used in other embodiments.

In one embodiment, the body 105 includes a battery 115 that powers the electrical system of the device 100, enabling the device 100 to be discharged when the trigger 120 is depressed and the safety 125 disengaged. The battery 115 may be removable, for example, to allow the battery to be replaced or charged via a separate charging mechanism. While a majority of the battery length may be positioned in a chamber inside the housing at the base of the body 105, a portion of the battery 115 may be positioned on an outside of the housing to allow a user (e.g., a clinician) to easily remove the battery 115 from the body 105. Once charged, the battery 115 may be reinserted into the chamber. In another embodiment, the battery 115 may be charged while engaged with the body 105, e.g., via a charging cable or other mechanism. While the battery 115 is shown as located at a base of the body 105, one of skill in the art will recognize that the battery could be positioned elsewhere on the device 100, such as in the handle.

The trigger 120 is located on a handle portion of the body 105 and controls the flow of pressurized gas into the device 100, causing activation of the pressure delivery system inside the housing when the trigger 120 is depressed. In one embodiment, the trigger is electrical and is driven by the battery 115, as discussed above. Alternatively, the trigger is mechanical and powered directly from wall power.

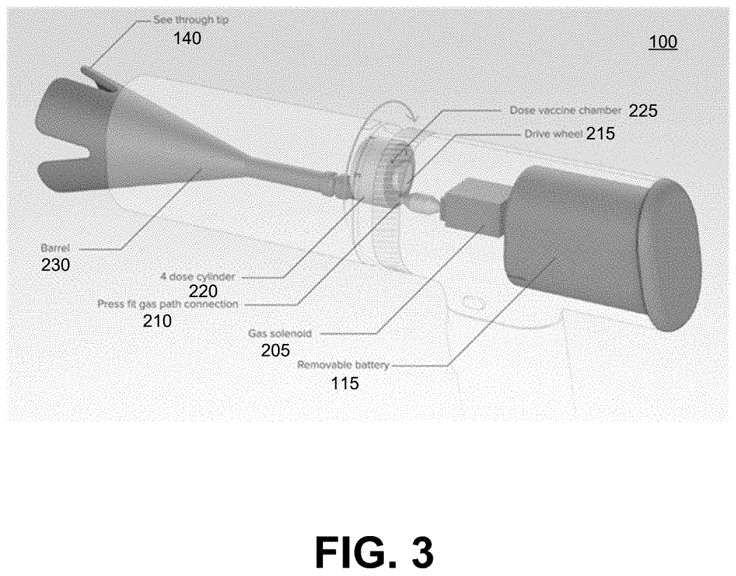

When activated, the trigger 120 causes a solenoid valve (shown and discussed below with respect to FIG. 3) to open, allowing input of pressurized gas via the gas connection 130 into the dose chamber. In one embodiment, the trigger 120 must be activated for at least ten milliseconds (ms) to achieve sufficient particle penetration and delivery pressure to release particles from the cartridge 135.

The trigger 120 is functional only when the safety 125 is disengaged. In one embodiment, the safety 125 is a button located at a top of the handle portion and is disengaged when pushed in. Once depressed, the safety 125 activates the trigger 120 for a specified period of time, e.g., ten seconds, thirty seconds, etc. If the device 100 is not discharged (i.e., the trigger 120 not depressed) within the specified time, the safety 125 is reengaged such that the user must press the safety 125 again to reactivate the trigger 120. In one embodiment, after the device 100 is discharged, there may be a delay (e.g., of N seconds) before the device 100 may be fired again.

The gas connection 130 is an inlet at the base of the handle that enables the device 100 to be connected to an external compressed gas source via a hose. As discussed below with respect to FIG. 3, activation of the trigger causes the input of pressurized gas obtained via the gas connection 130 through the solenoid valve and gas path connection into the dose chamber to allow the material in the chamber to be propelled through the barrel into the epidermis or mucosal tissue of the subject. In various embodiments, helium, nitrogen, or hydrogen gas may be used, however one of skill in the art will recognize that other pressurized gasses may be used in other embodiments. Additionally, in one embodiment, the gas tank is pressurized to approximately 1500 pounds per square inch (PSI). Alternatively, the gas tank is pressurized to above 500 PSI for 400 PSI delivery of the dose or to above 300 PSI for 200 PSI delivery.

The cartridge 135 may be coupled to the reusable body 105 via the cartridge release ring 110 and contain one or more doses of the material. As discussed below with respect to FIG. 3, an interior of the cartridge 135 includes a dose cylinder having a plurality of chambers, each configured to carry a dose of material for delivery to a subject. The cylinder is coupled to a first distal end of the elongated barrel 230 that spans the length of the cartridge 135 and protrudes outwardly from an opening in the cartridge. As shown in FIG. 2, the second distal end of the barrel comprises a nozzle 140 that may be placed against the subject (e.g., against the subject's arm or other skin sites) for delivery of the material into the epidermis. Alternatively, the device 100 may be used for mucosal tissue delivery of the material. The configuration of the nozzle 140 provides sufficient surface area to enable material penetration into the epidermis or mucosal tissue of the subject. Additionally, while the nozzle 140 shown in FIG. 2 is transparent, in other embodiments, the nozzle 140 is opaque.

FIG. 3 illustrates an internal view of the medical delivery device 100 of FIG. 1, according to one embodiment. In the embodiment shown in FIG. 3, the internal components of the medical delivery device 100 include the removable battery 115, a solenoid valve 205, a gas path connection 210, a drive wheel 215, a dose cylinder 220, a dose chamber 225, and a barrel 230.

The solenoid valve 205 opens and closes to control the flow of pressurized gas into the dose chamber. In a default state (i.e., when the trigger 120 is not depressed and/or the safety 125 is engaged), the solenoid valve 205 is closed such that pressurized gas does not enter the chamber 225 containing the dose of material. Activation of the trigger 120 and disengagement of the safety 125 activates the solenoid valve (i.e., causes the solenoid valve 205 to open) and permits the gas to enter the cartridge through the opening in the valve 205. The solenoid valve 205 remains open when the trigger 120 is depressed.

The solenoid valve 205 may be internally vented or externally vented. In one embodiment, use of an externally vented solenoid valve 205 lowers the rise time (i.e., the time from the opening of the solenoid valve 205 to achieve maximum pressure) as compared to a conventional internally vented valve. High-pressure gas flowing through the solenoid valve 205 causes the gold particles in the dose chamber 225 to become dislodged and begin to flow through the barrel 230. Accordingly, the rapid increase in pressure achieved with an externally vented solenoid valve 205 allows for optimal acceleration of the gold particles.

In an alternate configuration, a burst membrane is used with an internally vented solenoid valve 205 to control the flow of gas into the dose chamber 225. The burst membrane may be comprised of gas-impermeable aluminized mylar such that gas cannot pass into the chamber 225 until a pressure threshold is exceeded and the membrane has burst.

In various embodiments, the device 100 is operated under conditions ranging from 200-500 PSI. In a configuration in which approximately 400 PSI of supplied pressure is used, the device 100 delivers high-pressure gas flow with an average rise time of 2.30±0.08 ms to an average peak pressure of 309.44±5.98 PSI to enter the dose chamber 225. Such a pressure delivery profile allows for release of the material from the chamber 225 under high pressure conditions to achieve the required particle acceleration speeds for epidermal or mucosal tissue delivery and penetration. Upon release of the trigger 120, the solenoid valve 205 closes, and the pressure downstream of the valve 205 drops back down to 0 PSI. In one embodiment, maximum pressure is achieved when the solenoid valve 205 remains open for a time period greater than or equal to the rise time of approximately 2 ms.

Additionally, while the device 100 is standardly operated under conditions of an input pressure of 400 PSI, in other embodiments, the device 100 uses an operating pressure of 200 PSI due to enhanced particle acceleration resulting from the supersonic barrel 230, achieving a full particle release and delivery profile compared to 400 PSI. Operation of the device 100 at an input pressure of 200 PSI reduces the noise generated by the device 100 and provides compatibility with solenoid valves having different sizes and weights as compared to operation at a 400 PSI input pressure. In embodiments in which the input pressure is 200 PSI, the outlet pressure of the solenoid valve 205 is approximately 164.75±4.04 PSI with a rise time of approximately 2.29±0.23 ms. Additionally, in various embodiments, valves having varying opening mechanisms (direct or indirect), flow coefficients, and weights are used.

The gas path connection 210 is a chamber connecting the solenoid valve 205 to the dose chamber 225. When the solenoid valve 205 is open, the pressurized gas flows through the gas path connection 210 into the chamber 225.

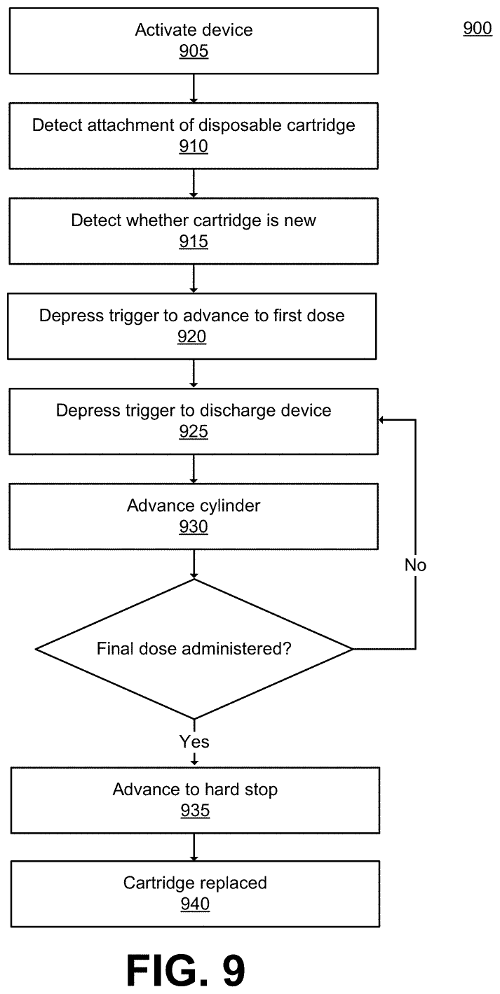

The drive wheel 215 is a chamber advancement mechanism on the handle-side of the device 100 that causes the dose cylinder 225 containing the material at the barrel-side to rotate after each dose is administered. In one embodiment, advancement of the cylinder 225 is automatic and not user-dependent. Operation of the drive wheel 215 is discussed below with respect to FIG. 9.

The dose cylinder 220 is located in the disposable cartridge 135 on the barrel-side of the device 100 adjacent to the drive wheel 215 inside the housing of the reusable body 105 on the handle-side. The dose cylinder 220 comprises a plurality of dose chambers 225 that each contain a single dose of the material. While the embodiment shown in FIG. 3 and described herein contemplates a four-dose cylinder, one of skill in the art will recognize that the cylinder 220 may contain additional or fewer chambers in other embodiments to enable delivery of different numbers of material doses. As discussed above and below, the drive wheel 215 causes the cylinder 220 to rotate to advance to a first dose chamber 225, to each subsequent chamber 225 after discharge, and to a hard stop after the final dose has been administered. As shown in FIG. 3, each chamber 225 is labeled with a corresponding dose number.

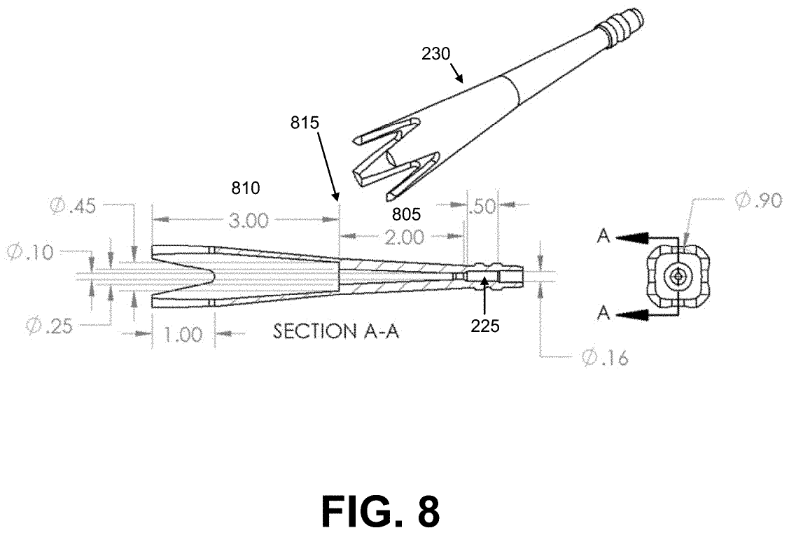

The barrel 230 (also referred to as a “supersonic barrel”) is positioned inside the disposable cartridge 135 and has an elongated body that extends from a first distal end where the barrel 230 is coupled to the cylinder 220 to a second distal end at an outlet of the device 100. The second distal end of the barrel 230 may be placed flush against the epidermis or mucosal tissue of the subject. The barrel 230 is shaped to allow particles from the dose chamber 225 and propelled by the pressurized gas to achieve at least a target velocity at the second distal end (i.e., for penetration into the epidermis or mucosal tissue). In one embodiment, the barrel 230 includes a primary expansion zone beginning at the first distal end and an overexpansion zone having a conical shape beginning at an approximate midpoint of the barrel 230 and expanding in diameter to the second distal end. Example specifications of the supersonic barrel are shown and discussed below with respect to FIG. 8.

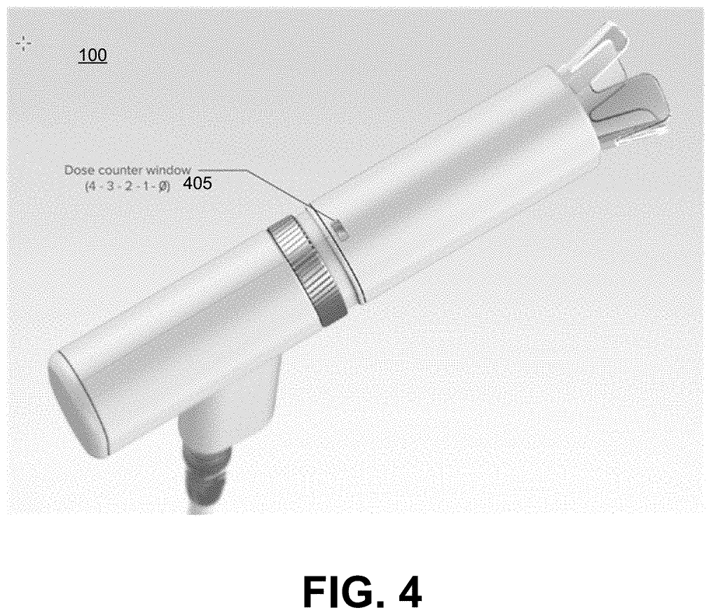

FIG. 4 illustrates a dose counter window 405 of the medical delivery device 100 of FIG. 1, according to one embodiment. In one embodiment, the window 405 comprises a cut-out in the housing of the disposable cartridge 135 such that the dose number located on the outside of each dose chamber 225 is viewable to the user (e.g., the clinician administering the dose), indicating a number of remaining doses of material available in the disposable cartridge 135. After each dose is administered and the cylinder 220 rotated to a subsequent dose chamber 225, the window 405 displays the updated number of available doses. Once the final dose has been administered, the counter window indicates that no remaining doses are available, such that the cartridge 135 may be discarded and replaced with a new cartridge 135 containing the same or different material.

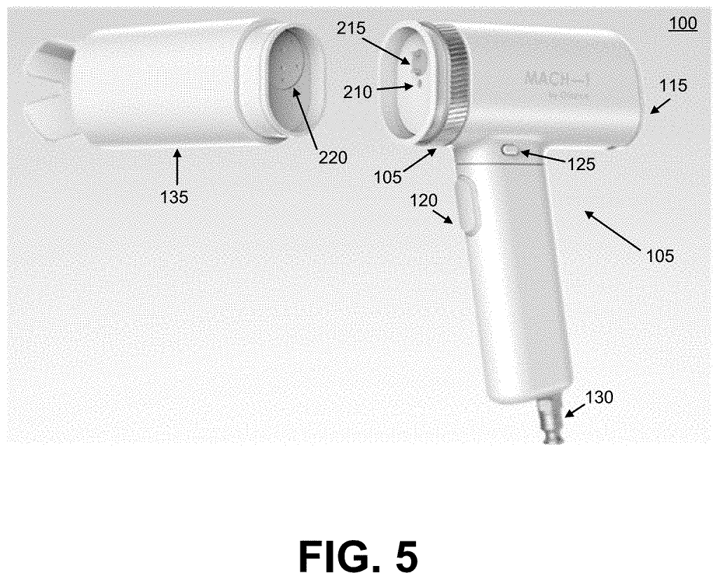

FIG. 5 illustrates a first exploded view of the separable housing of the medical delivery device 100 of FIG. 1, according to one embodiment. As shown in FIG. 5 and discussed above, the housing of the device 100 is separable into two portions for the replacement of the dose cartridge 135. Components located on the reusable body 105 include the cartridge release ring 105, battery 115, trigger 120, safety 125, gas connection 130, and drive wheel 215, which causes the dose cylinder 220 in the disposable cartridge 135 to turn after each dose is administered. Located below the drive wheel 215 in FIG. 5 is the outlet of the gas path connection 210. When the device 110 is assembled (i.e., the disposable cartridge 135 coupled to the reusable body 105 via the cartridge release ring 110), the outlet of the gas path connection 210 is positioned flush against a dose chamber 225 in the cylinder 220. Accordingly, in the embodiment shown in FIG. 5, a dose chamber 225 in the discharge position is the chamber 225 located at the bottom of the dose cylinder 220, and a dose of the material may be discharged from the cylinder 220 when the chamber 225 in which the dose is contained is rotated to the bottom of the cylinder 220. One of skill in the art will recognize that, in other configurations, the gas path connection 210 may be positioned elsewhere, such as above the drive wheel 215.

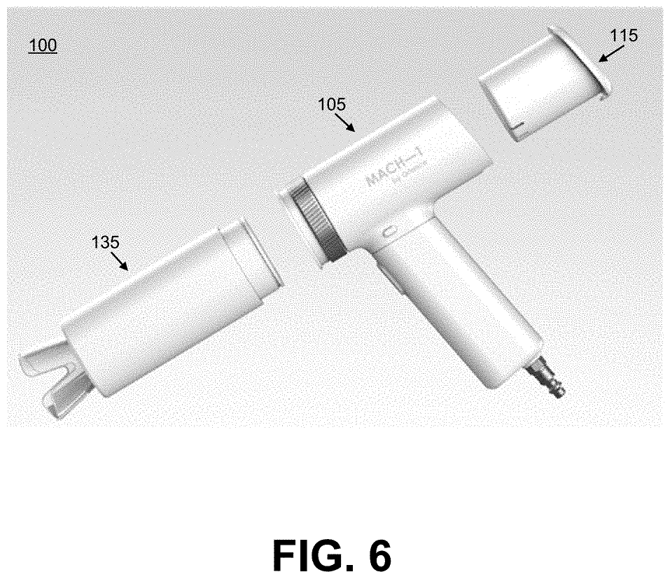

FIG. 6 illustrates a second exploded view of the separable housing of the medical delivery device 100 of FIG. 1, according to one embodiment. As shown in FIG. 6 and discussed above, the battery 115 may be removed from the device 100 for charging or replacement. In some embodiments, the trigger 120 is electrical and is driven by the battery 115. However, in other embodiments, the trigger 120 is mechanical and does not require power.

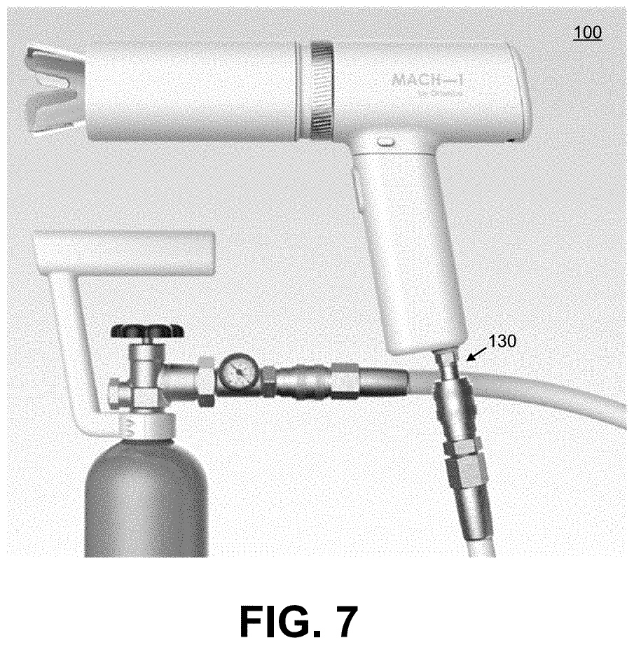

FIG. 7 illustrates the medical delivery device 100 of FIG. 1 connected to a compressed gas source, according to one embodiment. As shown in FIG. 7 and discussed above, the device 100 is connected to the gas source via a hose attached at a first end to the gas source and at a second end to the gas connection 130 on the device 100. In one embodiment, activation of the trigger 120 causes the input of pressurized gas obtained via the gas connection 130 through the solenoid valve 205 and gas path connection 210 into the dose chamber 225 to allow the material to be propelled through the barrel 230 into the epidermis or mucosal tissue of the subject

FIG. 8 illustrates example specifications of the supersonic barrel 230 of the medical delivery device 100 of FIG. 1, according to one embodiment. A target gas velocity through the barrel 230 is a supersonic velocity. Use of a supersonic barrel, such as the barrel 230, optimizes the density of gold delivered throughout the target to maximize intracellular delivery of particles into more cells while retaining high cell viability, with higher maximum particle deposition at input pressures of 200-500 PSI. The supersonic barrel 230 also improves gold particle penetration compared to legacy barrels.

As discussed above with respect to FIG. 3, the barrel 230 has an elongated body that extends from a first distal end where the barrel 230 is coupled to the cylinder 220 to a second distal end at an outlet of the device 100 that is placed against the subject for delivery of the material into the epidermis or mucosal tissue. The barrel 230 includes a primary expansion zone 805 beginning at the first distal end and an overexpansion zone 810 beginning at an approximate midpoint of the barrel 230. In one embodiment, the primary expansion zone 805 has a first inner diameter of approximately 0.08-0.12 inches at the first distal end, a second inner diameter of approximately 0.16-0.25 inches, and a length of approximately 1.6-3.0 inches. The overexpansion zone 810 has a first inner diameter of approximately 0.16-0.24 inches, a second inner diameter of approximately 0.56-0.84 inches, and a length of approximately 1.6-3.0 inches. In one example configuration, the second inner diameter of the primary expansion zone 805 is 0.25 inches, and the second inner diameter of the overexpansion zone is 0.75 inches.

In one embodiment, the overexpansion zone 810 has an inner-diameter expansion-to-length ratio that is higher than the inner-diameter expansion to length ratio of the primary expansion zone 805. For example, the overexpansion zone 810 inner-diameter expansion-to-length ratio may be twice or at least 1.5 times as high as the inner-diameter expansion to length ratio of the primary expansion zone 805.

The primary expansion zone 805 and the overexpansion zone 810 are separated by a step 815 that breaks the high velocity jet away from the wall of the barrel 230 in a clean fashion. In one embodiment, the step 815 is approximately 0.1 inches in length radially such that, when the final diameter of the primary expansion zone 805 is 0.25 inches, the diameter at the step 815 is 0.35 inches (0.25 inches at the terminal end of the primary expansion zone 805 plus 0.1 inches radial step). In this embodiment, the final diameter at the terminal end of the overexpansion zone 810 is 0.75 inches. The step 815 may have a constant diameter over its length and comprise an orifice that enables the downstream flow of particles to be free of boundary effects or conditions and allow the flow to be supersonic and separated from the inner wall of the barrel 230.

The dimensions of the supersonic barrel 230 discussed above are for example only. In alternate embodiments, the dimensions and ratios may be within 10-100% of the numbers listed above. The dimensions may also be proportionally scaled in various embodiments.

### Example Method

FIG. 9 is a flow chart illustrating a method 900 for operating the medical delivery device 100 of FIG. 1, according to one embodiment. In some embodiments, the operations in the method 900 are performed in a different order or can include different or additional steps.

In the embodiment shown in FIG. 9, the method 900 begins by activating 905 the device 100 (e.g., powering on the device 100 via an “on/off” switch or similar activation mechanism). At 910, a reed switch is used to detect whether a disposable cartridge 135 containing one or more doses of material is coupled to the reusable body 105. The device 100 cannot be discharged if no cartridge 135 is detected.

At 915, absolute position detection is used to verify the device position and detect whether a cartridge is “new.” As discussed above, each cartridge 135 is used with a single subject (e.g., patient), such that the cartridge 135 must be replaced if the device 100 is to be used to administer material to a different patient.

The trigger 120 is depressed 920 a first time to “purge” the device 100, causing the drive wheel 215 to advance the dose cylinder 220 to a first chamber 225 containing a dose of material. In embodiments in which the cartridge 135 contains four chambers 225 containing four doses of the material, the trigger 120 is depressed 925 four additional times to discharge the doses into the epidermis or mucosal tissue

After each discharge of the device 100, the drive wheel 215 advances 930 the dose cylinder 220 to a subsequent chamber 225. After the final dose is administered, the drive wheel 215 advances 935 to a hard stop that prevents the device 100 from firing, and the cartridge 135 is replaced 940.

### Additional Considerations

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments. This is done merely for convenience and to give a general sense of the disclosure. This description should be read to include one or at least one and the singular also includes the plural unless it is obvious that it is meant otherwise.

Upon reading this disclosure, those of skill in the art will appreciate still additional alternative structural and functional designs for a medical delivery device. Thus, while particular embodiments and applications have been illustrated and described, it is to be understood that the described subject matter is not limited to the precise construction and components disclosed herein and that various modifications, changes and variations which will be apparent to those skilled in the art may be made in the arrangement, operation and details of the method and apparatus disclosed. The scope of protection should be limited only by the following claims.

1. A medical delivery device, comprising:
a dose chamber configured to carry a dose of biological or non-biological material to be delivered to a subject;
an elongated barrel having a body that expands from a first distal end to a second distal end, the first distal end connected to the dose chamber and the second distal end being an outlet of the medical delivery device, the elongated barrel being shaped to allow a gas acceleration along the body to achieve at least a target gas velocity at the second distal end;
a gas inlet configured to be connected to an external compressed gas source used to propel the material to the subject through the elongated barrel;
a valve positioned between the dose chamber and the gas inlet, the valve configured to control a gas flow between the dose chamber and the gas inlet; and
a trigger controlling the valve.

2. The medical delivery device of claim 1, wherein the body of the elongated barrel is characterized as having a first expansion zone and a second over-expansion zone to allow the dose of material to accelerate from the first distal end, through the first expansion zone, the second over-expansion zone, to the second distal end, the first expansion zone having a first inner-diameter expansion to length ratio and the second expansion zone having a second inner-diameter expansion to length ratio, the second inner-diameter expansion to length ratio being higher than the first inner-diameter expansion to length ratio.

3. The medical delivery device of claim 2, wherein a target gas velocity through the elongated barrel is at a supersonic velocity.

4. The medical delivery device of claim 2, wherein the second inner-diameter expansion to length ratio is at least twice as high as the first inner-diameter expansion to length ratio.

5. The medical delivery device of claim 2, wherein the second inner-diameter expansion to length ratio is at least 1.5 times as high as the first inner-diameter expansion to length ratio.

6. The medical delivery device of claim 2, wherein the first expansion zone has a first inner diameter of 0.08″-0.12″ at the first distal end and a second inner diameter of 0.16″-0.25″ fully expanded, and the first expansion zone has a length of 1.6″-3″. The medical delivery device of claim 2, wherein the second over-expansion zone has a first inner diameter of 0.16″-0.24″ a first end connected to the first expansion zone and a second inner diameter of 0.56″-0.84″ at the second distal end, and second over-expansion zone has a length of 1.6″-3″.

8. The medical delivery device of claim 1, wherein the medical delivery device is configured to be operated at an input pressure at or above 400 PSI.

9. The medical delivery device of claim 8, wherein the valve is configured to be opened to allow the gas communication between the dose chamber and the gas inlet for at least 10 ms after the trigger is activated.

10. The medical delivery device of claim 1, wherein the medical delivery device is configurable for use with an internally-vented solenoid valve, an externally-vented solenoid valve, or an internally-vented solenoid valve with a burst membrane.

11. The medical delivery device of claim 1, further comprising a multi-dose cylinder containing two or more dose chambers, each chamber configured to carry a dose of material.

12. The medical delivery device of claim 11, wherein the multi-dose cylinder contains four dose chambers.

13. The medical delivery device of claim 1, further comprising a drive wheel configured to advance the multi-dose cylinder after a dose is administered.

14. A method for operating a medical delivery device, the method comprising:
activating the medical delivery device;
detecting attachment of a disposable cartridge to a reusable body of the medical delivery device, the disposable cartridge comprising a multi-dose cylinder containing two or more dose chambers, each chamber configured to carry a dose of biological or non-biological material for delivery to a subject;
verifying a starting position of the multi-dose cylinder;
advancing the multi-dose cylinder to a first dose chamber;
discharging the medical delivery device to deliver the dose contained in the first dose chamber through an elongated barrel in the disposable cartridge to a subject;
advancing the multi-dose cylinder to a subsequent dose chamber; and
after discharge of each dose of material contained in a dose chamber of the multi-dose cylinder, advancing the multi-dose cylinder to a hard stop such that the medical delivery device cannot be discharged unless a replacement disposable cartridge is attached to the reusable body.

15. The method of claim 14, wherein the medical delivery device is configured to be connected to an external compressed gas source used to propel the material through the elongated barrel.

16. The method of claim 15, wherein a target gas velocity through the elongated barrel is at a supersonic velocity.

17. The method of claim 15, wherein the elongated barrel is characterized as having a first expansion zone and a second over-expansion zone to allow the dose of material to accelerate from the first distal end, through the first expansion zone, the second over-expansion zone, to the second distal end, the first expansion zone having a first inner-diameter expansion to length ratio and the second expansion zone having a second inner-diameter expansion to length ratio, the second inner-diameter expansion to length ration being higher than the first inner-diameter expansion to length ratio.

18. The method of claim 14, wherein the multi-dose cylinder contains four dose chambers.

19. The method of claim 14, wherein the medical delivery device further comprises a drive wheel configured to advance the multi-dose cylinder.

20. The method of claim 14, wherein the medical delivery device is configurable for use with an internally-vented solenoid valve, an externally-vented solenoid valve, or an internally-vented solenoid valve with a burst membrane.
